(19) United States
(12) Patent Application Publication
(10) Pub. No.: US 2010/0330208 A1
(43) Pub. Date: Dec. 30, 2010

(54) APPARATUS AND PROCESS FOR EXTRACTION OF CONSTITUENTS FROM ALTERNATIVE MEDICINAL MATERIAL

(76) Inventor: Kam Wah Vasco Cheung, Hong Kong (CN)

Correspondence Address: WILKINSON & GRIST, 6TH FLOOR, PRINCE''S BUILDING, CHATER ROAD, CENTRAL, HONG KONG, HK

(21) Appl. No.: 12/870,859

(22) Filed: Aug. 30, 2010

Related U.S. Application Data
(63) Continuation of application No. 11/397,834, filed on Apr. 3, 2006.

(51) Int. Cl. A61K 36/00; B01D 11/02

(52) U.S. Cl. 424/725; 422/277; 422/128

(57) ABSTRACT

An apparatus and process as a system for extraction of constituents from alternative medicinal material is provided. The apparatus includes an outer container containing a solvent liquid, an inner container mounted within the outer container containing an alternative medicinal material, a microwave generator for supplying microwaves to the solvent liquid and the alternative medicinal material, and a motor for rotating the inner container within the outer container. The inner container includes a perforated wall, such that the alternative medicinal material in the inner container can be fully or partially immersed in the solvent liquid.

This application is a continuation of prior application Ser. No. 11/397,834, filed Apr. 3, 2006.

### FIELD OF THE INVENTION

The present invention relates to an apparatus and a process as a system for the extraction of constituents from alternative medicinal material.

### BACKGROUND

Traditionally, medications made from alternative medicinal material involve the process of slowly heating the material in a suitable solvent liquid such as water or alcohol, so as to break down the material to form a drink or a liquid with extracted constituents. Such traditional methods have generally been performed by using apparatus such as pots or similar vessels on a stove.

### SUMMARY

An apparatus for extraction of constituents from alternative medicinal material is provided. In one aspect, the apparatus includes an outer container and an inner container. The outer container defines a first volume adapted to receive a solvent liquid. The inner container is mounted within the outer container and defines a second volume adapted to receive the alternative medicinal material. The inner container includes a wall defining a perforation positioned to permit the solvent liquid to flow between the first volume and second volume. The apparatus further includes a microwave generator adapted to supply microwaves to at least a portion of the second volume and a motor for rotating said inner container within said outer container.

In one embodiment, the outer container includes an outer wall and an inner wall. The microwave generator is disposed between the outer and inner walls, and the microwaves are adapted to be emitted through a window of the inner wall.

In one embodiment, the frequency of the microwaves is in the range of about 2,400 MHz to about 2,500 MHz.

In one embodiment, an ultrasound generator for generating ultrasound vibration to the solvent liquid and the alternative medicinal material is provided.

In one embodiment, the microwave generator includes a magnetron.

In one embodiment, the inner container includes a sieve.

A process for extraction of constituents from the alternative medicinal material is also provided. Initially, the alternative medicinal material is supplied in a motor-driven inner container which is rotatably mounted within an outer container. A solvent liquid is then supplied to the outer container, such that the alternative medicinal material in the inner container can be fully or partially immersed in the solvent liquid. Further, microwaves are supplied to the inner portion of the inner container to irradiate the solvent liquid and the alternative medicinal material. Finally, the inner container is rotated.

Optionally, ultrasound can be supplied to the solvent liquid and the alternative medicinal material.

The inner container can be alternately rotated in clockwise and/or anticlockwise directions at a speed of about 10 rpm. Liquid extract can be drained from the outer container by spinning the inner container at a high speed after the process for the extraction is over.

### BRIEF DESCRIPTION OF THE DRAWINGS

Specific embodiments of the invention will now be described by way of example with reference to the accompanying drawings wherein:

FIG. 1 is a schematic view of an extractor for the extraction of constituents from alternative medicinal material;

FIG. 2 is a cross-sectional view of the extractor;

FIG. 3 is a perspective view of a inner container of the extractor; and

FIG. 4 is a flow chart of the extracting process in accordance with an embodiment of the present invention.

### DETAILED DESCRIPTION

Reference will now be made in detail to certain embodiments of the invention, examples of which are also provided in the following description. Exemplary embodiments of the invention are described in detail, although it will be apparent to those skilled in the relevant art that some features that are not particularly important to an understanding of the invention may not be shown for the sake of clarity.

Furthermore, it should be understood that the invention is not limited to the precise embodiments described below and that various changes and modifications thereof may be effected by one skilled in the art without departing from the spirit or scope of the invention. For example, elements and/or features of different illustrative embodiments may be combined with each other and/or substituted for each other within the scope of this disclosure and appended claims.

In addition, improvements and modifications which may become apparent to persons of ordinary skill in the art after reading this disclosure, the drawings, and the appended claims are deemed within the spirit and scope of the present invention.

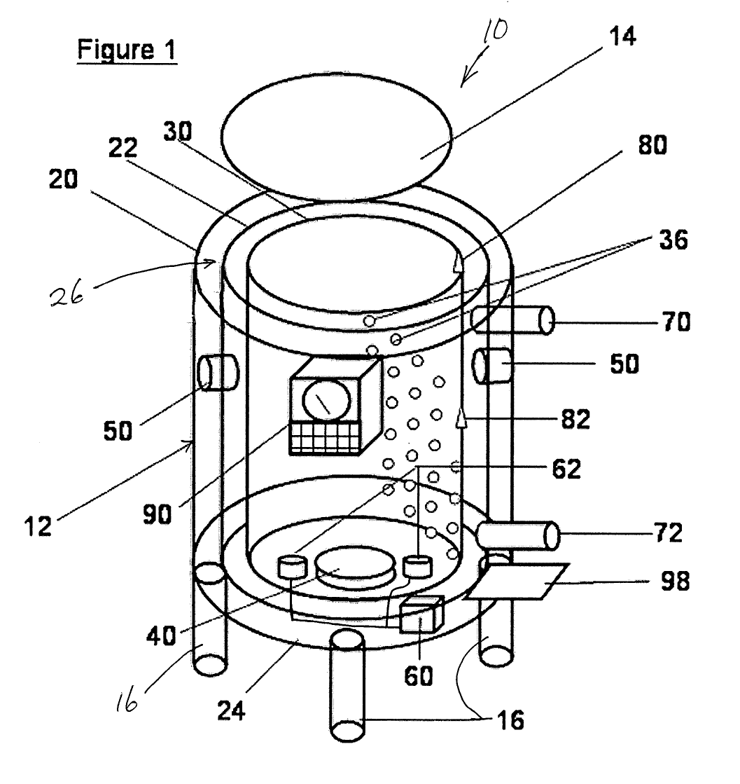

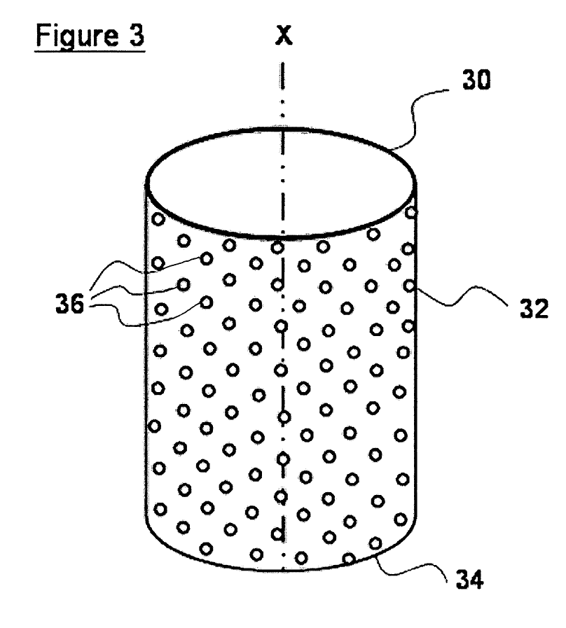

Referring now to the drawings, in which like reference numerals represent like parts throughout the drawings, FIGS. 1, 2 and 3 are schematic and cross-sectional views of an extractor 10, respectively. The extractor 10 is adapted to be used for the extraction of constituents from alternative medicinal material. As used herein, the term “alternative medicinal material” refers to herbal material and/or other medicinal material which can be used as alternative medicine (e.g., Chinese medicine) components. Such alternative medicinal material may be in fresh, dry, and/or processed forms. The extractor 10 is programmable, portable and suitable for household or clinic use.

The extractor 10 includes an outer container 12, a lid 14 adapted to cover the outer container 12, and optionally a plurality of legs 16 for supporting the outer container 12 on a surface or ground. In the illustrated embodiment, the lid 14 is hingedly connected to the outer container 12. The outer container 12 and the lid 14, when closed, define an enclosure 18 in which extracting process takes place. The outer container 12 is adapted to receive therein solvent liquid such as water for the preparation of liquid extract from the alternative medicinal material. Other suitable solvent liquid, such as alcohol, may be used.

In the illustrated embodiment, the outer container 12 includes an outer wall 20, an inner wall 22 and a bottom wall 24. The outer and inner walls 20, 22 can be cylindrical or other shapes. The outer and inner walls 20, 22 may be disposed coaxially with respect to each other defining therebetween an annular space 26. The outer and inner walls 20, 22 and the bottom wall 24 may be made of stainless steel or any other suitable material. In another embodiment, the outer container 12 may include an inner wall and a bottom wall, but not the outer wall.

An inner container 30 may be rotatably mounted within the outer container 12 in a coaxial relationship with the outer and inner walls 20, 22. The inner container 30 can be cylindrical or other shapes. The inner container 30 is adapted to receive therein alternative medicinal material. The inner container 30 has a wall 32 defining at least one perforation positioned to permit the solvent liquid in the outer container 12 flows into the inner space of the inner container 30 through the perforation. As such, the alternative medicinal material in the inner container 30 can be fully or partially immersed in the solvent liquid. In the illustrated embodiment, the inner container 30 is a sieve which includes an array (for example, about 9×14) of apertures 36. The aperture 36 can be circular and have a diameter of about 5 mm. The inner container 30 may be made of Poly Tetra Fluoro Ethylene (PTFE) or any other suitable material.

The outer container 12 and the inner container 30 may be of any appropriate sizes. In one embodiment, the diameter of the cylindrical outer wall 20, the cylindrical inner wall 22, and the cylindrical inner container 30 may be about 60 cm, 38 cm, and 27 cm respectively. The height of the inner container 30 may be about 30 cm.

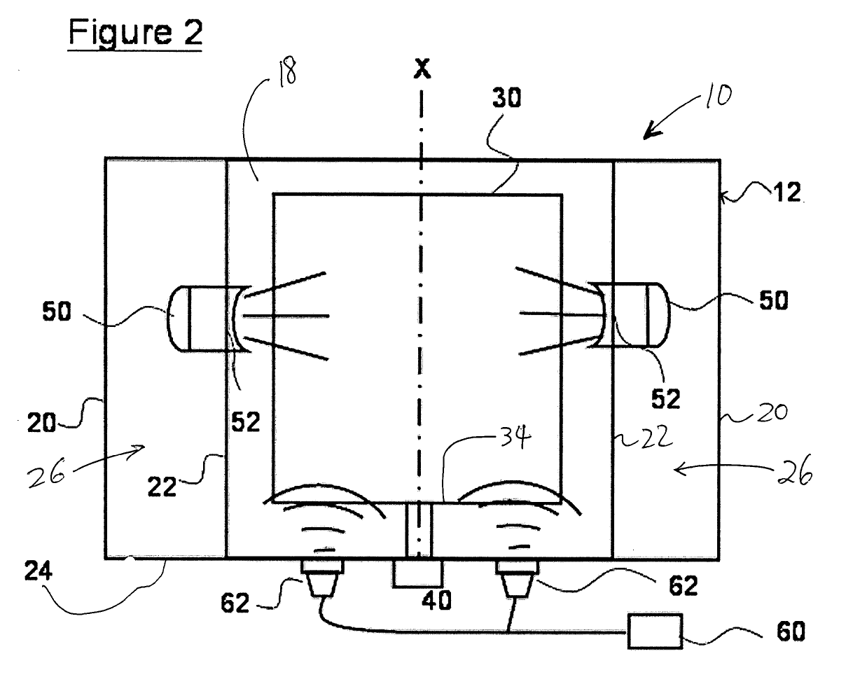

The extractor 10 includes a motor 40 for rotating the inner container 30 within the outer container 12 in either clockwise or anticlockwise direction about a central axis X, as shown in FIG. 2. The motor 40 may be mounted to the bottom wall 24 of the outer container 12 and operatively coupled to the bottom wall 34 of the inner container 30.

One or more microwave generators, such as magnetrons, are provided. In the illustrated embodiment, there are two magnetrons 50 provided on the outer container 12. The two magnetrons 50 may be disposed between the outer and inner walls 20, 22 of the outer container 12 at opposite sides thereof. The two magnetrons 50 are employed to supply microwaves to the outer container 12. The microwaves generated by the two magnetrons 50 are adapted to be emitted through windows 52 provided on the inner wall 22 of the outer container 12. The magnetron 50 (e.g. Hitachi 2M231J) can generate microwaves having a frequency in the range of about 2,400 MHz to about 2,500 MHz and have a power output of about 800 W or about 1600 W. The microwaves generated are used as a heat source to heat up the solvent and extracted constituents in the liquid. The number of microwave generators may vary depending on the size of the outer container. For example, the extractor may have only one microwave generator.

Optionally, an ultrasound generator 60 may be employed to generate ultrasound vibration to the solvent liquid and the alternative medicinal material in the outer container 12. In the illustrated embodiment, the ultrasound generator 60 is provided at the bottom of the outer container 12. The ultrasound generator 60 may include one or more ultrasound transducers 62. In the illustrated embodiment, two ultrasound transducers 62 are attached to the bottom wall 24 of the outer container 12. The ultrasound generator 60 may have a power output power ranging from about 300 W to about 1000 W, and the frequency of the ultrasound vibration can be about 22K. The ultrasound vibration creates a cavitation effect during the extraction of the constituents from the alternative medicinal material. The cavitation cracks the constituents so that the extraction process can be speeded up.

The extractor 10 may also include a solvent liquid inlet for supplying solvent liquid thereto and a liquid extract outlet for draining of liquid extract therefrom. In the illustrated embodiment, the solvent liquid inlet 70 is provided at an upper portion of the outer container 12, and a liquid extract outlet 72 is provided at a lower portion of the outer container 12.

The extractor 10 may further include a control panel 90 for controlling the extracting process. The control panel 90 may have a liquid level selection switch for selecting the amount of solvent liquid, a microwave power control unit for selecting the microwave power, a time control unit for setting the extraction time, and/or an ultrasound switch for turning the ultrasound vibration on or off. The liquid level switch in an embodiment of the invention is connected to a liquid level control unit. There are a wide variety of suitable control units suitable for this purpose known to those skilled in the art. For example, a liquid level control unit may be a feedback controller configured to open a valve to permit liquid flow into the extractor 10 until a level sensor (see below) indicates that a particular liquid level selected by the level selection switch has been reached. The control unit then closes the valve in response. As an alternative, the control unit may be configured to discharge a predetermined amount of liquid into the extractor 10 by measuring the flow rate (e.g., via the use of the flow meter) and permit liquid flow until the integral reaches a particular value selected by the level selection switch. Other switches are linked to their respective controllers, which may be any of the wide variety of controllers suitable for the respective purposes.

The extractor 10 may also include a lid sensor 80, a liquid level sensor 82, and/or a microwave sensor (not shown). An error message can be prompted to alert a user in case the lid 14 is not properly closed, the solvent liquid level is not at a proper level, and/or microwave leakage is detected. The extractor 10 may also include a temperature control unit for controlling the temperature inside the outer container 12.

It is to be understood that the extractor 10 does not have to be positioned where the axis X is perpendicular to the ground. The extractor 10 can be positioned where the axis X is parallel to the ground or in an angle with respect to the ground.

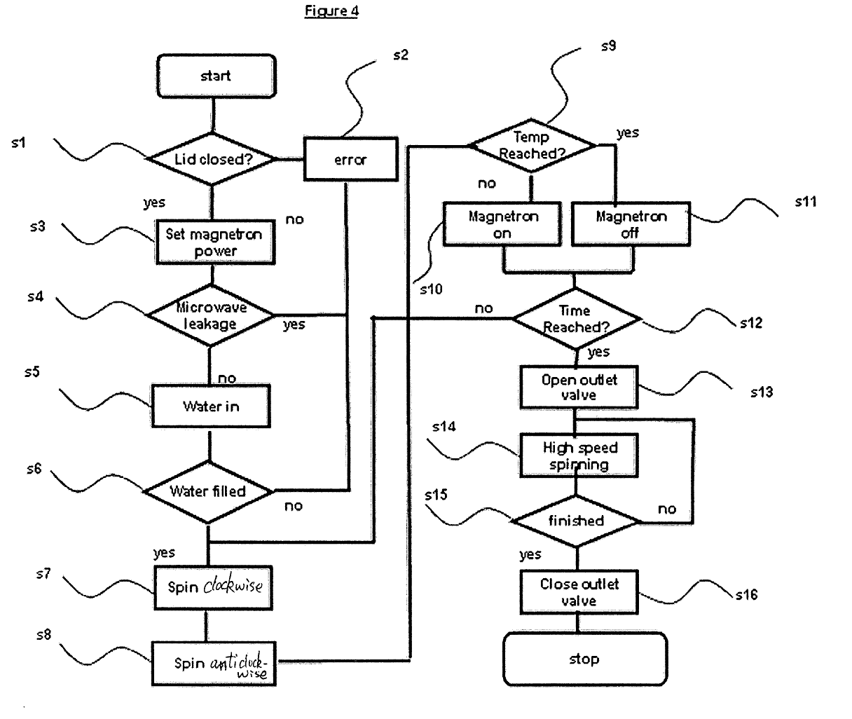

A process for the extraction of constituents from alternative medicinal material will be described hereinbelow with reference to FIG. 4.

The user initially places a desired amount of the alternative medicinal material within the inner container 30. Depending on the size of the inner container 30, the amount of the alternative medicinal material for extraction may be in the range of about 100 gm to about 1 kg.

The user may then select the microwave power. In the illustrated embodiment, the use of either one or both of the magnetrons 50 (S3) can be selected. If the user selects low microwave power, only one of the two magnetrons 50 will be in operation. If the user selects high microwave power, both magnetrons 50 will be in operation. The microwave sensor can be used to detect microwave leakage (S4). If there is microwave leakage, an error message appears in order to alert the user (S2).

The user may then select the amount of solvent liquid to be used in the extracting process (S5). Depending on the size of the outer container 12 and the amount of alternative medicinal material inside the inner container 30, the amount of solvent liquid to be supplied to the outer container 12 may be in the range of about 1 liter to about 10 liters. The alternative medicinal material may be fully or partially immersed into the solvent liquid during the extracting process. The ratio of the alternative medicinal material and the solvent liquid may vary depending on the prescription. For example, the alternative medicinal material to solvent liquid ratio may be 1:6. The water level sensor 82 is adapted to check the level of solvent liquid inside the outer container 12 (S6). If the solvent liquid is not properly filled or reached a minimum level, an error message appears so as to alert the user (S2).

The user may select the processing time of the extracting process, and may select ultrasound vibration, if desired.

It is to be understood that the steps of placing the alternative medicinal material in the inner container 30, selecting the microwave power, selecting the amount of solvent liquid, and selecting the processing time of the extracting process can be in any order.

The user then starts the extracting process. The selected amount of solvent liquid is then supplied to the outer container 12 through the solvent liquid inlet 70.

The lid 14 is generally required to be closed after the extracting process is started. The lid sensor 80 is adapted to check whether the lid 14 is properly closed (S1). If the lid 14 is not properly closed, an error message appears in order to alert the user (S2).

During the extracting process, the inner container 30 may alternately rotate in a clockwise direction (S7) and an anticlockwise direction (S8) at a speed of about 10 rpm. Alternatively, the inner container 30 may rotate only in a clockwise direction or only in an anticlockwise direction. The temperature control unit is adapted to check the temperature inside the outer container 12 (S9). The magnetron or magnetrons 50 will be turned off if the temperature reaches a set value (S10, S11).

Depending on the time selected by the user, the extracting process stops after reaching the end of the selected processing time (S12). The processing time can be preset, e.g., for about 5 to 30 minutes, depending on the amount and the types of the alternative medicinal material. After the extracting process has stopped, a buzzer may be sound to alert the user.

To collect the liquid extract from the outer container 12, the user can open the liquid extract outlet 72 (S13). When the outlet 72 is opened, the motor 40 may be activated thereby spinning the inner container 30 at a high speed (such as 1000 rpm) in either clockwise or anticlockwise direction (S14). The liquid extract within the outer container 12 will then be drained from the outer container 12 through the liquid extract outlet 72 into a cup. A filter (not shown) may be used to filter the liquid extract before it enters the liquid extract outlet 72. A cup holder 98 may be employed to hold the cup in a proper position. The spinning and draining process may stop after a preset period of time (such as one minute) (S15) and the liquid extract outlet 72 will be closed (S16).

The extracting process can be stopped at any time. The process can also be resumed after stop.

There may also be a process for cleaning the extractor 10. Cleaning can be achieved by supplying water to the outer container 12, spinning the inner container 30 for a preset period of time (such as one minute), and draining the water from the outer container 12. This cleaning step may be repeated several times.

Using microwaves and ultrasound vibration, the method for extracting constituents from alternative medicinal material of the present invention is faster than traditional methods using pots and stove. Comparing to the traditional methods, the method for extracting constituents from alternative medicinal material is easier to use and control because the extractor of the present invention is programmable by a control panel.

While the present invention has been shown and described with particular references to a number of preferred embodiments thereof, it should be noted that various other changes or modifications may be made without departing from the scope of the present invention.

What is claimed is:

1. A system for extraction of constituents from alternative medicinal material comprising:
a solvent liquid;
an alternative medicinal material;
an outer container defining a first volume containing said solvent liquid;
an inner container mounted within said outer container and defining a second volume containing said alternative medicinal material, said inner container comprising a wall defining a perforation positioned to permit said solvent liquid to flow between said first volume and said second volume;
a microwave generator adapted to supply microwaves to at least a portion of the second volume; and
a motor for rotating said inner container.

2. The system of claim 1, wherein said inner container comprises a sieve.

3. The system of claim 1, wherein said outer container comprises an outer wall and an inner wall, said microwave generator is disposed between said outer and inner walls, and said microwaves is adapted to be emitted through a window of said inner wall.

4. The system of claim 1, wherein the frequency of said microwaves is in the range of about 2,400 MHz to about 2,500 MHz.

5. The system of claim 1, further comprising an ultrasound generator for generating ultrasound vibration to said solvent liquid and said alternative medicinal material.

6. The system of claim 5, wherein said ultrasound generator comprises at least one ultrasound transducer attached to said outer container.

7. The system of claim 5, wherein the frequency of said ultrasound generator is about 22K.

8. The system of claim 1, further comprising solvent liquid inlet for supplying solvent liquid thereto.

9. The system of claim 1, further comprising a liquid extract outlet for draining of liquid extract therefrom.

10. The system of claim 1, further comprising a lid adapted to cover said outer container.

11. The system of claim 1, further comprising a liquid level sensor.

12. The system of claim 1, further comprising a control panel, said control panel having a liquid level selection switch for selecting the amount of solvent liquid, a microwave power control unit for selecting microwave power, and/or a time control unit for setting the time of extraction, the liquid level selection switch operatively connected to a liquid level controller adapted to fill the solvent liquid in the outer container to a level selected by the liquid level selection switch.

13. The system of claim 1, wherein said microwave generator comprises a magnetron.

14. A process for extraction of constituents from alternative medicinal material, comprising:
supplying said alternative medicinal material in a motor-driven inner container rotatably mounted within an outer container;
supplying a solvent liquid to said outer container, such that said alternative medicinal material in said inner container can be fully or partially immersed in said solvent liquid;
supplying microwaves to an inner portion of the inner container to irradiate said solvent liquid and said alternative medicinal material; and
rotating said inner container.

15. The process of claim 14, further comprising the step of supplying ultrasound to said solvent liquid and said alternative medicinal material.

16. The process of claim 14, wherein supplying microwaves and rotating the inner container are performed substantially simultaneously for about 5 minutes to about 30 minutes.

17. The process of claim 14, wherein rotating the inner container comprises rotating the inner container alternately in clockwise and/or anticlockwise directions at a speed of about 10 rpm.

18. The process of claim 14, further comprising the step of draining liquid extract from said outer container after the rotating step.

19. The process of claim 18, wherein draining said liquid extract from said outer container comprises spinning said inner container at a high speed.
